(19) United States
(12) Patent Application Publication
(10) Pub. No.: US 2008/0319280 A1
(43) Pub. Date: Dec. 25, 2008

(54) Implantable Biotelemetry Device

(75) Inventors: Jason August, Toronto (CA); Robert Griffin, Richmond Hill (CA); John K. Stevens, Stratham, NH (US); Paul Waterhouse, Copetown (CA)

Correspondence Address: MICHAEL J. BUCHENHORNER, 8540 S.W. 83 STREET, MIAMI, FL, 33143, US

(73) Assignee: Visible Assets Inc., Mississauga (CA)

(21) Appl. No.: 12/057,358

(22) Filed: Mar. 27, 2008

Related U.S. Application Data
(60) Provisional application No. 60/908,896, filed on Mar. 29, 2007.

(51) Int. Cl. A61B 5/00

(52) U.S. Cl. 600/301

(57) ABSTRACT

An implantable device for in vivo monitoring of biotelemetry data includes: a waterproof housing completely encasing the implantable device, the waterproof housing constructed from a material with chemical and fatigue resistance plus thermal stability for placement in a living being; a radio frequency modem located inside the housing and operable at a low radio frequency not exceeding one megahertz; an antenna located within the housing and operatively coupled with the radio frequency modem; a fully programmable microprocessor located within the housing and operatively coupled with the modem; at least one sensor located within the housing for detecting the biotelemetric data; a memory; and a connector for connecting to a power source to power the programmable microprocessor.

### CROSS-REFERENCE TO RELATED APPLICATIONS

This application is a non-provisional of, and claims priority from, commonly-owned, co-pending U.S. patent application Ser. No. 60/908,896, filed on Mar. 29, 2007, “Implantable Biotelemetry Device,” which is incorporated by reference as if fully set forth herein.

### FIELD OF THE INVENTION

The present invention relates to the field of implantable monitoring devices and more particularly relates to an active low frequency (LF), inductive radiating radio transceiver tag in a subcutaneous device for providing biotelemetry data.

### BACKGROUND OF THE INVENTION

### RFID Background

Radio Frequency IDentification (RFID) tags and telemetry for implantable devices have a long history. Several of the early issued patents do not specify the frequency for the preferred embodiment. Studies have shown that the frequency can change the radio tag's ability to operate in harsh environments, near liquids, or conductive materials, as well as the tag's range, power consumption, and battery life.

Transmissions from commercial RFID tags are impeded by any steel and water contained in a tissue (high frequency HF reduced by 50% and UHF 100%) and a passive low frequency (LF) tag would have a range of only a few inches. Steel and other conductive metals may de-tune the antennas and degrade performance.

Many Electronic Article Surveillance (EAS) systems function using a back-scattered non-radiating mode and most are also inductive frequencies. Many other telemetry systems in widespread use for pacemakers, implantable devices, and sensors in rotating centrifuges also make use of this back-scattered mode to reduce power consumption. Low frequencies (myriametric) can transmit through conductive materials and work in harsh environments. Most of these implantable devices also use the back-scattered communication mode for communication to conserve battery power.

Accordingly, more recent and modem RFID tags are passive, back-scattered transponder tags and have an antenna consisting of a wire coil or an antenna coil etched or silk screened onto a personal computer (PC) board. These tags use a carrier that is reflected back from the tag. The carrier is used by the tag for four functions:

The carrier contains the incoming digital data stream signal, in many cases the carrier only performs the logical function to turn the tag on/off and activate the transmission of its ID. In other cases, the data may be a digital instruction. The carrier serves as the tag's power source. The tag receives a carrier signal from a base station and uses the rectified carrier signal to provide power to the integrated circuitry and logic on the tag. The carrier serves as a clock and time base to drive the logic and circuitry within the integrated circuit. In some cases, the carrier signal is divided to produce a lower clock speed.

The carrier may also serve as a frequency and phase reference for radio communications and signal processing. The tag can use one coil to receive a carrier at a precise frequency and phase reference for the circuitry within the radio tag for communications back through a second coil to the reader/writer, making accurate signal processing possible.

However, the major disadvantage of the back-scattered mode radio tag is that it has limited power, limited range, and is susceptible to noise and reflections over a radiating active device. This is largely because the passive tag requires a minimum of one volt on its antenna to power the chip, not because of loss of communication signal. As a result, many back-scattered tags do not work reliably in harsh environments and require a directional “line of site” antenna.

One method to extend the range of a passive back-scattered tag has been to add a thin, flat battery to the back-scattered tag so the power drop on the antenna is not the critical range limiting factor. However, since all of these tags use high frequencies, the tags must continue to operate in back-scattered mode to conserve battery life. The power consumed by any electronic circuit tends to be related to the frequency of operation.

Thus, most recent active RFID tags that have a battery to power the tag circuitry are active tags and devices operating in the 13.56 MHz to 2.3 GHz frequency range, and also work as back-scattered transponders. Because these tags are active back-scattered transponders, they cannot work in an on-demand peer-to-peer network setting, plus they may require line of sight antennas that provide a carrier that “illuminates” an area or zone or an array of carrier beacons.

It is also generally assumed that high frequency (HF) or ultra high frequency (UHF) passive back-scattered transponder radio tags will have a lower cost to manufacture than an LF passive back-scattered transponder because of the antenna. An HF or UHF tag can obtain a high Q, 1/10 wavelength antenna by etching or conductive silver silk screening the antenna geometry onto a flexi-circuit. An LF (30 to 300 KHz) or ULF (300-3000 Hz) antenna cannot use either because the Q will be too low due to high resistance of the traces or silver paste. Therefore, LF and ULF tags must use wound coils made of copper.

Finally, active radiating transceiver tags require large batteries and are expensive, perhaps costing up to hundreds of dollars. The transmission speed is inherently slow using ultra low frequency (ULF) as compared to HF and UHF since the tag must communicate with low baud rates because of the low transmission carrier frequency. Many sources of noise exist at these ULF frequencies from electronic devices, motors, fluorescent ballasts, computer systems, and power cables. Thus, ULF is often thought to be inherently more susceptible to noise. Radio tags in this frequency range are considered more expensive since they require a wound coil antenna because of the requirement for many turns to achieve optimal electrical properties (maximum Q). In contrast, HF and UHF tags can use antennas etched directly on a printed circuit board. ULF would also have even more serious distance limitations with such an antenna. Current networking methods used by high frequency tags, as used in HF and UHF, are impractical due to such low bandwidth of ULF tags described above.

Implantable telemetry systems are known in the field of medical science. The most common forms of these systems are pacemakers, drug delivery systems, and defibrillators. Most have relied either on high frequency or low frequency backscattered modes of operation, and in many cases wired or short range systems have been proposed. These devices generally rely upon remote sensors. This poses significant problems. Remote sensors often require specialized catheters and it is a challenge to keep them in place.

In addition, the implant is susceptible to infiltration by body fluids at the point where the required electrical leads join the remote sensors to the internal circuitry within the implantable device. The body fluids will disable the electrical circuits within the device.

There is a need for an implantable device for monitoring biotelemetry that requires no remote sensors and is impervious to bodily fluids. Therefore, there is a need for a sensor system that overcomes the foregoing shortcomings the prior art.

### SUMMARY OF THE INVENTION

Briefly, according to an embodiment of the invention, an implantable device for in vivo monitoring of biotelemetry data includes: a waterproof housing completely encasing the implantable device, the waterproof housing constructed from a material with chemical and fatigue resistance plus thermal stability for placement in a living being; a radio frequency modem located inside the housing and operable at a low radio frequency not exceeding one megahertz; an antenna located within the housing and operatively coupled with the radio frequency modem; a fully programmable microprocessor located within the housing and operatively coupled with the modem; at least one sensor located within the housing for detecting the biotelemetric data; a memory; and a connector for connecting to a power source to power the programmable microprocessor.

### BRIEF DESCRIPTION OF THE DRAWINGS

FIG. 1a shows an implantable device, according to an embodiment of the present invention;

FIG. 1b shows a side view of the device of FIG. 1a, according to the embodiment of the present invention shown in FIG. 1a;

FIG. 1c shows a comparative view of the device of FIG. 1a, according to the embodiment of the present invention;

FIG. 2 shows an illustration of the interior of the device of FIG. 1a, according to the embodiment of the present invention;

FIG. 3 shows another embodiment wherein a reader is worn on a belt, according to an embodiment of the present invention; and

FIG. 4 shows the battery and electrical components of the device of FIG. 1a, according to the embodiment of the present invention.

### DETAILED DESCRIPTION

We describe a RuBee® radio tag as an implantable device for monitoring of biotelemetry data using the low-frequency RuBee® protocol. Radio tags communicate via magnetic (inductive communication) or electric radio communication to a base station or reader, or to another radio tag. RuBee® radio tags function through water and other bodily fluids, and near steel, with an eight to fifteen foot range, a five to ten-year battery life, and three million reads/writes. These tags operate at 132 Khz and are full on-demand peer-to-peer, radiating transceivers.

RuBee® is a bidirectional, on-demand, peer-to-peer transceiver protocol operating at wavelengths below 450 Khz (low frequency). A transceiver is a radiating radio tag that actively receives digital data and actively transmits data by receiving power for an antenna. A transceiver may be active or passive. The RuBee® standard is documented in the IEEE Standards body as IEEE P1902.1™. Encasing a RuBee® radio tag in an implantable bio-compatible housing produces a cost effective medical device for chronic monitoring of blood pressure, flow, temperature, PH, glucose and other biotelemetry data, with local memory and long range RuBee® telemetry. This device can be advantageously used in humans, livestock and pets.

Low frequency (LF), active radiating transceiver tags are especially useful for visibility and for tracking objects with large area loop antennas over other more expensive active radiating transponder high frequency (HF)/ultra high frequency (UHF) tags. These LF tags will function in harsh environments, near water and steel, and may have full two-way digital communications protocol, digital static memory and optional processing ability, sensors with memory, and ranges of up to 100 feet. The active radiating transceiver tags can be far less costly than other active transceiver tags (many under one dollar), and often less costly than passive back-scattered transponder RF-ID tags, especially those that require memory and make use of EEPROM. With an optional on-board crystal, these low frequency radiating transceiver tags also provide a high level of security by providing a date-time stamp, making full AES encryption and one-time based pads possible.

The main advantage of the RuBee® tags is that they can transmit well through water and other bodily fluids and near steel. This is because RuBee® operates at a low frequency. Low frequency radio tags are immune to nulls often found near steel and liquids, as in high frequency and ultra high-frequency tags. This makes them ideally suited for use in implantable devices where previously the corrosive effects of bodily fluids had posed significant problems. In fact, tests have shown that the RuBee® tags work well even when fully submerged in water. This is not true for any frequency above 1 MHz. Radio signals in the 13.56 MHz range have losses of over 50% in signal strength as a result of water, and anything over 30 MHz have losses of 99%. In addition, as the frequency goes up the power required to operate the implant also increases, so battery life is reduced.

RuBee® networks operate at long-wavelengths and accommodate low-cost radio tags at ranges to 100 feet. The standard, IEEE P1902.1, “RuBee Standard for Long Wavelength Network Protocol”, will allow for networks encompassing thousands of radio tags operating below 450 KHz.

Form factor

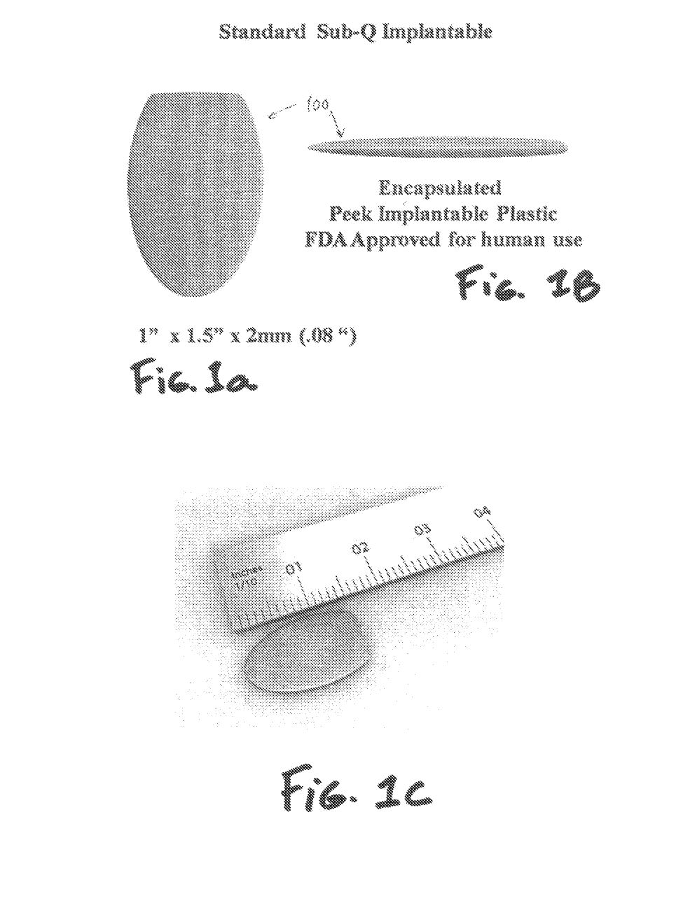

Referring to FIG. 1a there is shown an embodiment of the present invention. The RuBee®-enabled implantable radio tag device 100 is housed in a container constructed of a bio-compatible material. The container is preferably constructed from a long-term implantable plastic, such as polyetheretherketones (PEEK). We describe an embodiment housed in PEEK, a high temperature resistant engineered thermoplastic with excellent chemical and fatigue resistance plus thermal stability. The PEEK container is FDA approved for use in humans. Although PEEK plastic is recommended, any FDA-approved long-term thermoplastic that is insoluble to all common solvents could be used.

The device 100 as shown has an ovoid shape, but a circular or rectangular shape may also be used. Because this device 100 is meant to be implanted in a living being, it is of necessity small in size. FIG. 1b shows a side view of the device 100. The width of the device 100 at its thickest portion is only approximately 2 mm. FIG. 1c shows a comparative view of the device 100 juxtaposed against a ruler. As can be seen the device 100 measures approximately 1 inch by 1½ inches.

Components

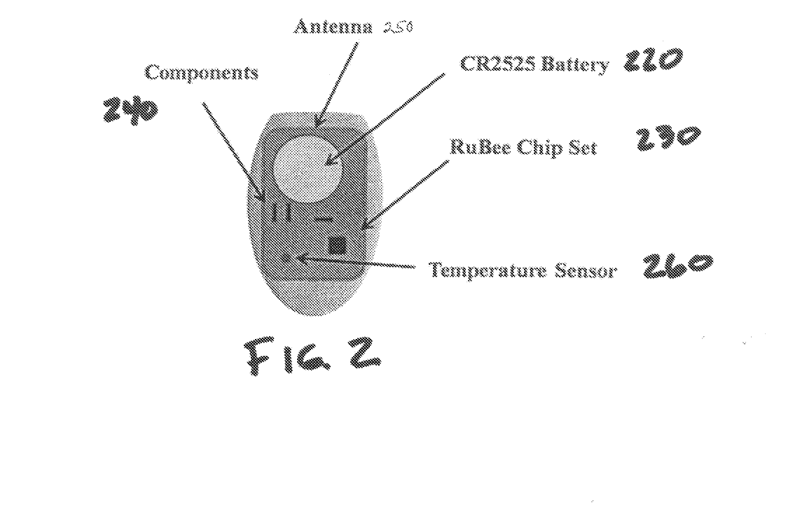

Referring to FIG. 2 there is shown an illustration of the components of the device 100. We show a CR2525 battery 220, a RuBee® chip set 230, electrical components 240, an antenna 250 and a temperature sensor 260. The battery 220 is a lithium battery approximately the size of an American quarter-dollar with a five to ten year life and up to three million read/writes. Note that a microprocessor may be used rather than the chip set 230 shown in this one example.

Sensors

The number of sensors and the type of sensors depend on the intended use of the device 100. It is assumed that the sensors are physiological parameter sensors and/or activity parameter sensors. The physiological parameter sensors detect biotelemetry data such as respiration rate, blood oxygen saturation level, temperature, blood pressure, pH, length of a Q-T interval, length of a P-R interval, thoracic impedance changes, nerve activity, and biochemical concentrations such as enzymes and glucose. Additionally, pulse oximetry sensors for providing differential measurements of arterial blood flow may also be used. The activity parameter sensors detect motion and acceleration. Other sensors not mentioned here may be advantageously used within the spirit and scope of the invention.

The sensors for measuring blood pressure may be of the MEMS type (microelectrical mechanical systems) which respond to pressure differences by altering the value of an electrical property of the MEMS value of the device (such as capacitance or resistance).

For some medical uses, a photodetector may be used as a sensor. In those cases, the device housing will include a translucent or transparent “window.” In another embodiment of the invention, the device 100 may include sensors that are capacitively or resistively coupled to fluid and tissue within the living being in which the device 100 is implanted.

Antenna

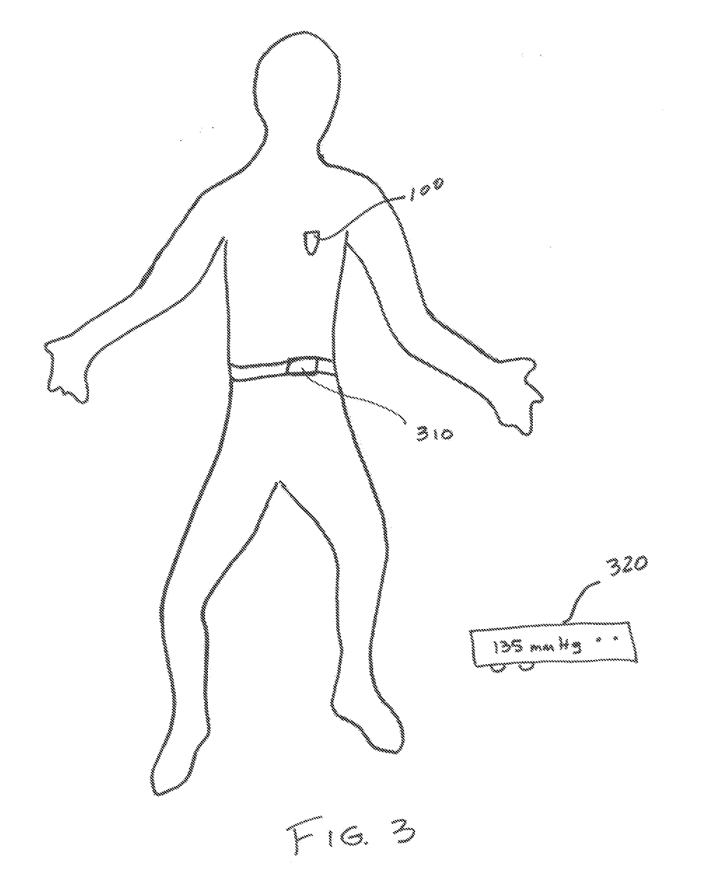

The antenna 250 shown in FIG. 2 is a small loop antenna with a range of eight to fifteen feet. A reader or monitor may be placed anywhere within that range in order to read the sensor(s). As shown in FIG. 3 the reader 310 may be attached to a belt with a small display on top 320 to indicate status with optional buttons on the side to control display operation.

Electrical Components

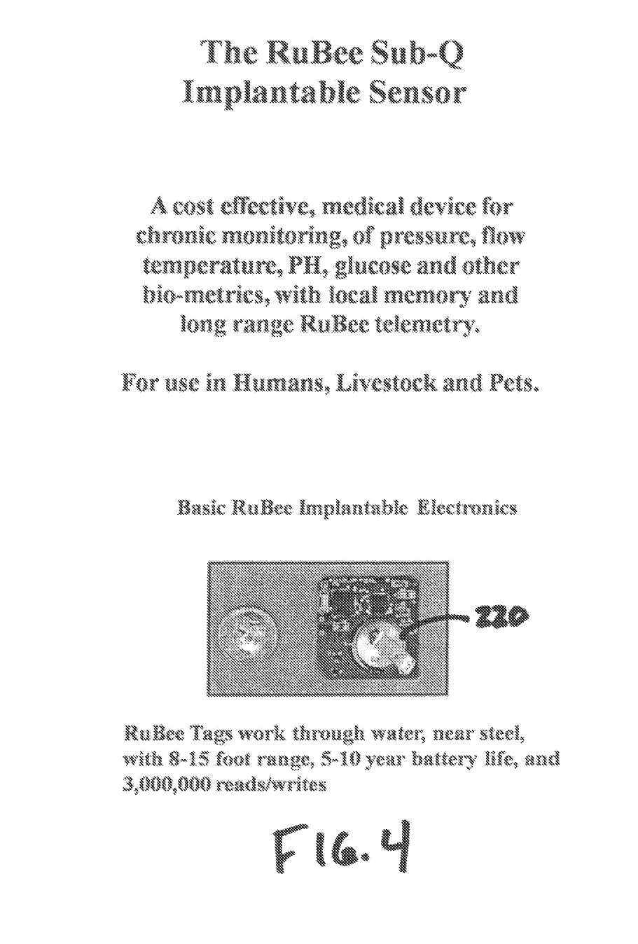

Note that the electrical components 240 are housed within the body of the device 100 and are completely enclosed within the device 100 when the device is sealed. See FIG. 4 for an illustration of the electrical components 240 and the battery 220. Housing the electrical components and the sensors within the same sealed enclosure eliminates the problem of fluid infiltration caused by the use of leads described earlier.

Implementation

The device 100 operates by monitoring in vivo at least one physiological parameter, and transmitting data received from the at least one sensor to a receiver located outside of the patient. The data can then be analyzed. The data may be stored in static memory as a data log and harvested once a day, or may be stored as a histogram in the static memory. Note that because the RuBee® radio tag 100 is a transceiver, data can also be written to the tag 100. The RuBee® tag 100 may be read by a small, low power “belt reader,” worn by a patient, or by a low frequency area reader placed anywhere within a room. Other optional embodiments will be described below.

The active low frequency tags may use amplitude modulation, or in some cases, phase modulation, and can have ranges of many tens of feet up to one hundred feet with the use of a loop antenna. The active tags include a battery, and a microprocessor or chip set. Optionally, a crystal may be included for time stamps. The combination of the crystal or clock and the sensors serve to provide a temporal history of status events. For example, if a sensor operable to detect fluctuations in blood pressure detects a sudden increase in pressure and emits a warning picked up by the processor, the blood pressure reading can be logged with a timestamp. Associating status events with a date/time provides much more valuable information than the status event by itself.

The sensors as described earlier, due to their low power requirements at these low radio frequency transmission frequencies (below 1 mHz), allow continuous monitoring of these biological characteristics while a patient carries out his/her daily activities. The sensor data can be continuously monitored in real-time and/or stored in a memory device for subsequent analysis by the treating physician. The patient can go about his/her daily activities while the sensors capture and record biotelemetry data such as systolic blood pressure and flow. For example, this stored data can be used to monitor a patient's blood pressure after endovascular repairs, such as abdominal aortic aneurysm (AAA) and to prevent thoracic embolisms.

The inductive mode of the RuBee® tag uses low frequencies, 3-30 kHz VLF or the Myriametric frequency range, 30-300 kHz LF in the Kilometric range, with some in the 300-3000 kHz MF or Hectometric range (usually under 450 kHz). Since the wavelength is so long at these low frequencies, over 99% of the radiated energy is magnetic, as opposed to a radiated electric field. Because most of the energy is magnetic, antennas are significantly (10 to 1000 times) smaller than ¼ wavelength or 1/10 wavelength, which would be required to efficiently radiate an electrical field. This is the preferred mode.

As opposed to the inductive mode radiation above, the electromagnetic mode uses frequencies above 3000 kHz in the Hectometric range, typically 8-900 MHz, where the majority of the radiated energy generated or detected may come from the electric field, and a ¼ or 1/10 wavelength antenna or design is often possible and utilized. The majority of radiated and detected energy is an electric field.

### Embodiments

In one embodiment an antenna is attached to the outside of the patient to pick up the signals from the device 100. This can be used to indicate real-time status of the sensors and indicate a fault condition. This embodiment may be used in conjunction with the belt reader shown in FIG. 3.

In another possible embodiment a small antenna (e.g., 3″×4″) is placed on the monitor itself. This antenna may be optionally in the same plane as the coil antenna 250 in the device 100.

In another embodiment a reader that has been web-enabled is attached to an antenna about 12×17 inches and placed in a room where a patient wearing the implantable device 100 is located. In this case the patient does not have to wear a monitor 310 and the implantable device 100 may be read from a distance without help or cooperation from the patient. This may be the ideal situation for use in livestock and other animals.

In yet another embodiment a large loop antenna may be placed around the room where the person (or animal) wearing the implant 100 is located. The large loop antenna can be connected to a reader in a router/base station. In this case the reader is optimally tuned for this specific loop of about 8′ by 16′ and the loop can be draped on the floor around the room. The router/base station connects the room to a network to allow for remote monitoring.

Therefore, while there have been described what are presently considered to be the preferred embodiments, it will be understood by those skilled in the art that other modifications can be made within the spirit of the invention.

We claim:

1. An implantable device for in vivo monitoring of biotelemetry data, the implantable device comprising:
a waterproof housing completely encasing the implantable device, the waterproof housing constructed from a material with chemical and fatigue resistance plus thermal stability that is insoluble to all common solvents for placement in a living being;
a radio frequency modem operable at a low radio frequency not exceeding one megahertz, said radio frequency modem comprising a full duplex transmitter and receiver;
an antenna located operatively coupled with the radio frequency modem;
a fully programmable microprocessor operatively coupled with the radio frequency modem, and operable to transmit data received from the at least one sensor;
at least one sensor operatively coupled with the microprocessor for detecting the biotelemetric data;
a memory located operatively coupled with the programmable microprocessor, said memory comprising identification data for identifying the implantable device; and
a connector configured for connecting to a power source to power the programmable microprocessor,
wherein the radio frequency modem, the antenna, the microprocessor, the at least one sensor, the memory and the connector are all located within the housing.

2. The implantable device of claim 1 wherein the waterproof housing is approximately one inch by one and one-half inches in size.

3. The implantable device of claim 1 wherein the waterproof housing is ovoid in shape.

4. The implantable device of claim 1 wherein the material for the waterproof housing is an engineered thermoplastic.

5. The implantable device of claim 4 wherein the engineered thermoplastic comprises polyetheretherketones.

6. The implantable device of claim 1 wherein the radio frequency modem is configured to transmit and receive at a low radio frequency less than 450 kilohertz.

7. The implantable device of claim 6 wherein the radio frequency modem is configured to transmit and receive at a low radio frequency less than 312 kilohertz.

8. The implantable device of claim 1 wherein the identification data further comprises identifying data about the living being.

9. The implantable device of claim 1 wherein the living being is a human.

10. The implantable device of claim 1 wherein the at least one sensor is a physiological parameter sensor that detects biotelemetry data selected from a group consisting of: blood pressure, blood flow, body temperature, PH, glucose, respiration rate, blood oxygen saturation level, length of Q-T interval, length of a P-R interval, thoracic impedance changes, nerve activity, and biochemical concentrations such as enzymes and glucose.

11. The implantable device of claim 10 wherein the at least one sensor is a pulse oximetry sensor that provides differential measurements of arterial blood flow.

12. The implantable device of claim 1 wherein the at least one sensor is an activity parameter sensor that detects motion and acceleration.

13. The implantable device of claim 1 wherein the at least one sensor is a photo detector and the waterproof housing comprises a translucent window through which light can pass.

14. The implantable device of claim 10 wherein the at least one sensor may be capacitively coupled with fluid and tissue within the living being.

15. The implantable device of claim 1 further comprising the power source.

16. The implantable device of claim 1 wherein the microprocessor transmits a message responsive to a warning emitted by the at least one sensor.

17. The implantable device of claim 1 wherein the antenna comprises thin wire wrapped around an outside edge of an inside of the housing of the implantable device.

18. The implantable device of claim 1 further comprising a crystal that provides a timestamp for an event as detected by the at least one sensor.

19. The implantable device of claim 1 further comprising leads attached to said implantable device.

20. A system for in vivo monitoring of biotelemetry data for a living being, the system comprising:
a biotelemetry device implanted in the living being, said biotelemetry device operable to communicate via radio communication with a monitor and with another biotelemetry device, and comprising:
a waterproof housing completely encasing the implantable device, the waterproof housing constructed from a material with chemical and fatigue resistance plus thermal stability and that is insoluble to all common solvents for placement in a living being;
a radio frequency modem operable at a low radio frequency not exceeding one megahertz, said radio frequency modem comprising a full duplex transmitter and receiver;
an antenna operatively coupled with the radio frequency modem;
at least one sensor that detects biotelemetry data;
a fully programmable microprocessor operatively coupled with the radio frequency modem, and operable to transmit data received from the at least one sensor;
a memory operatively coupled with the programmable microprocessor, said memory comprising identification data for identifying the implantable device; and
a connector for a power source to power the programmable microprocessor; and
a monitor comprising a display for presenting the biotelemetry data detected by the at least one sensor.

21. The system of claim 20 further comprising a base station in communication with the biotelemetry device.

22. The system of claim 21 further comprising a computer operatively configured with the base station.

23. The system of claim 20 further comprising a belt reader worn by the living being.

24. The system of claim 20 further comprising a handheld reader.

22. The system of claim 20 further comprising a loop antenna placed around the proximity of the living being.

23. The system of claim 22 wherein the loop antenna is operatively coupled with a base station.

24. The system of claim 20 further comprising a data storage location for storing data transmitted from the biotelemetry device.
